(19) United States
(12) Patent Application Publication
(10) Pub. No.: US 2025/0375609 A1
(43) Pub. Date: Dec. 11, 2025

(54) INTRAVAGINAL DEVICE FOR TREATING PELVIC PAIN

(71) Applicant: CORNELL UNIVERSITY, Ithaca, NY (US)

(72) Inventor: Tamatha FENSTER, Ithaca, NY (US)

(21) Appl. No.: 18/877,209

(22) PCT Filed: Jul. 5, 2023
(86) PCT No.: PCT/US2023/069634

§ 371 Date: Dec. 19, 2024

Related U.S. Application Data
(60) Provisional application No. 63/358,813, filed on Jul. 6, 2022.

(51) Int. Cl. A61N 1/05; A61N 1/36

(52) U.S. Cl. CPC A61N 1/0524; A61N 1/36007

(57) ABSTRACT

An intravaginal device capable of supplying electrical stimulation is provided. In an example, the intravaginal device may be used to implement the TENS technique by stimulating nerves while positioned within a user's vagina. While positioned within the vagina, the provided medical device may stimulate the hypogastric nerve complex around the uterosacral ligaments. In some cases, the provided medical device may both apply electrical stimulation to treat pain and control bleeding during a menstrual cycle. For example, at least a portion of the provided medical device may include absorbent material, such as a tampon.

### PRIORITY CLAIM

The present application claims priority to and the benefit of U.S. Provisional Application 63/358,813, filed Jul. 6, 2022, the entirety of which is herein incorporated by reference.

### TECHNICAL FIELD

The present application relates generally to medical devices. More specifically, the present application provides an intravaginal device for treating pelvic pain.

### BACKGROUND

Chronic pelvic pain is a medical condition experienced by a portion of the female population globally that can significantly affect everyday activities. Some causes of chronic pelvic pain include gynecological adenomyosis, endometriosis, leiomyoma, adnexal mass, ovarian remnant syndrome, pelvic adhesions, vestibulitis, and vulvodynia. In some instances, the chronic pain can cause depression, anxiety, and/or sleep disturbances. Typical treatments for chronic pelvic pain include over-the-counter (OTC) pain relievers, nerve pain medication, opioid pain relievers, and interventional procedures. OTC pain relievers are rarely effective at treating chronic pelvic pain. Nerve pain drugs have significant side effects. Opioids not only have significant side effects (e.g., constipation, nausea, interference with clear thinking) but can also be dangerously addictive as they lose their effectiveness over time by sensitizing nerve pathways thereby making pain worse. Interventional procedures (e.g., innervation to uterus) have proven to have limited benefit and low patient satisfaction and agreement (especially for implanted devices) along with an inherent risk of the procedure. A need therefore exists for a device and method to better treat chronic pelvic pain.

### SUMMARY

The present application provides new and innovative medical devices and methods for treating chronic pelvic pain. More specifically, an intravaginal device capable of stimulating nerves while positioned within the vagina is provided. For instance, when in use, the provided medical device may stimulate the hypogastric nerve complex around the uterosacral ligaments. The provided medical device may be used to apply the TENS technique. In at least one example, the provided medical device is, or is included with (e.g., attached to), a tampon. In such an example, the provided medical device may both apply electrical stimulation to treat pain and control bleeding during a menstrual cycle.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular.

The present application describes various exemplary embodiments of intravaginal medical devices and methods for treating chronic pelvic pain. The application, however, is not limited to these exemplary embodiments and applications or to the manner in which the exemplary embodiments and applications operate or are described herein, but rather includes other embodiments and applications that can be contemplated by a person of ordinary skill in the art in view of the exemplary embodiments and applications disclosed in the present application.

In a first aspect, a medical device includes a body; at least one electrode integral with, or coupled to, the body; and a power source configured to supply the at least one electrode with electricity. The body is configured such that the body may be positioned within a vagina of a user.

In a second aspect, which may be combined with other aspects described herein (e.g., the 1st aspect), the body includes an absorbent material.

In a third aspect, which may be combined with other aspects described herein (e.g., the 1st aspect or the 2nd aspect), the body includes a tampon.

In a fourth aspect, which may be combined with other aspects described herein (e.g., the 1st aspect through the 3rd aspect), the medical device further includes a deployer, and the body includes a plurality of arms configured to interact with the deployer such that, prior to activation of the deployer, the plurality of arms are in a closed position, and subsequent to activation of the deployer, the plurality of arms are in an open position. The plurality of arms include the at least one electrode.

In a fifth aspect, which may be combined with other aspects described herein (e.g., the 5th aspect), the plurality of arms are arranged such that, in the open position, the plurality of arms span across the uterosacral ligaments of the user while the medical device is positioned within the vagina of the user.

In a sixth aspect, which may be combined with other aspects described herein (e.g., the 1st aspect through the 3rd aspect), the medical device further includes a deployer, and the body includes a cone-shaped portion configured to interact with the deployer such that, prior to activation of the deployer, the cone-shaped portion is in a closed position, and subsequent to activation of the deployer, the cone-shaped portion is in an open position. The cone-shaped portion includes the at least one electrode.

In a seventh aspect, which may be combined with other aspects described herein (e.g., the 1st aspect through the 6th aspect), the medical device further includes a deployer configured such that activating the deployer activates the power source to begin supplying electricity to the at least one electrode.

In an eighth aspect, which may be combined with other aspects described herein (e.g., the 1st aspect through the 7th aspect), the body is shaped such that the at least one electrode contacts the hypogastric nerve plexus of the user while the medical device is positioned within the vagina of the user.

In a ninth aspect, which may be combined with other aspects described herein (e.g., the 1st aspect through the 8th aspect), the medical device further includes (1) a transceiver or (2) a transmitter and a receiver.

In a tenth aspect, which may be combined with other aspects described herein (e.g., the 1st aspect through the 9th aspect), the medical device further includes an applicator, and the body is positioned within the applicator.

In an eleventh aspect, which may be combined with other aspects described herein (e.g., the 10th aspect), the applicator includes a first portion and a second portion configure to slide within the first portion, and the body is positioned within the first portion.

In a twelfth aspect, which may be combined with other aspects described herein (e.g., the 11th aspect), the body of the medical device includes a first end and a second end, and wherein the body is configured such that, upon deploying the medical device from the first portion, the first and second ends translate away from one another in a plane perpendicular to an axis along which the second portion is configured to slide.

In a thirteenth aspect, which may be combined with other aspects described herein (e.g., the 2nd aspect through the 12th aspect), a method for treating pelvic pain includes positioning a medical device within a vagina of a user; and applying transcutaneous electrical stimulation to the hypogastric nerve plexus of the user via the medical device positioned within the vagina of the user. The medical device includes a body configured such that it may be positioned within the vagina of the user, at least one electrode integral with, or coupled to, the body, and a power source configured to supply the at least one electrode with electricity.

In a fourteenth aspect, which may be combined with other aspects described herein (e.g., the 13th aspect), the method further includes absorbing menses of the user via the medical device positioned within the vagina of the user.

In a fifteenth aspect, which may be combined with other aspects described herein (e.g., the 13th aspect through the 14th aspect), applying transcutaneous electrical stimulation includes applying electrical pulses at a frequency within a range of 1 to 20 cycles per second.

In a sixteenth aspect, which may be combined with other aspects described herein (e.g., the 13th aspect through the 14th aspect), applying transcutaneous electrical stimulation includes applying electrical pulses at a frequency within a range of 80 to 120 cycles per second.

In a seventeenth aspect, which may be combined with other aspects described herein (e.g., the 13th aspect through the 16th aspect), the body includes a plurality of arms movable between a closed position and an open position, and the plurality of arms include the at least one electrode. In the fourteenth aspect, positioning the medical device includes: inserting the medical device into the vagina while the plurality of arms are in the closed position, and deploying the plurality of arms into the open position subsequent to the medical device being inserted into the vagina.

In an eighteenth aspect, which may be combined with other aspects described herein (e.g., the 17th aspect), positioning the medical device further includes positioning the plurality of arms in the open position such that the plurality of arms span across uterosacral ligaments of the user.

In a nineteenth aspect, which may be combined with other aspects described herein (e.g., the 13th aspect through the 18th aspect), the body includes a cone-shaped portion movable between a closed position and an open position, and the cone-shaped portion includes the at least one electrode. In the sixteenth aspect, positioning the medical device includes: inserting the medical device into the vagina while the cone-shaped portion is in the closed position, and deploying the cone-shaped portion into the open position subsequent to the medical device being inserted into the vagina.

In a twentieth aspect, which may be combined with other aspects described herein (e.g., the 19th aspect), positioning the medical device further includes positioning the cone-shaped portion in the open position such that the cone-shaped portion surrounds a cervix of the user.

In a twenty-first aspect, which may be combined with other aspects described herein (e.g., the 13th aspect through the 20th aspect), the medical device further includes a deployer, and the transcutaneous electrical stimulation is applied in response to activating the deployer.

In a twenty-second aspect, which may be combined with other aspects described herein (e.g., the 13th aspect through the 21st aspect), positioning the medical device includes positioning the medical device within the vagina such that the at least one electrode contacts the hypogastric nerve plexus of the user.

In a twenty-third aspect, which may be combined with other aspects described herein (e.g., the 10th aspect through the 22nd aspect), the transcutaneous electrical stimulation is applied in response to receiving a signal transmitted wirelessly from a device separate from the medical device.

In a twenty-fourth aspect, which may be combined with other aspects described herein (e.g., the 13th aspect through the 23rd aspect), positioning the medical device within the vagina of the user includes deploying the medical device from an applicator.

In a twenty-fifth aspect, which may be combined with other aspects described herein (e.g., the 24th aspect), the body of the medical device includes a first end and a second end, and wherein the body is configured such that, upon deploying the medical device, the first and second ends translate away from one another in a plane perpendicular to a central axis of the applicator.

Additional features and advantages of the disclosed device and method are described in, and will be apparent from, the following Detailed Description and the Appendix. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

A further understanding of the nature and advantages of the present disclosure may be realized by reference to the following drawings. In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

FIG. 1 illustrates a box diagram of a medical device, according to an aspect of the present disclosure.

FIG. 2A illustrates a side view of a first example implementation of the medical device of FIG. 1 in a closed position, according to an aspect of the present disclosure.

FIG. 2B illustrates a side view of the medical device of FIG. 2A in a deployed position, according to an aspect of the present disclosure.

FIG. 2C illustrates a cross section at plane A-A of the medical device of FIG. 2B, according to an aspect of the present disclosure.

FIG. 3A illustrates a side view of a second example implementation of the medical device of FIG. 1 in a closed position, according to an aspect of the present disclosure.

FIG. 3B illustrates a side view of the medical device of FIG. 3A in a deployed position, according to an aspect of the present disclosure.

FIG. 4 illustrates the first example implementation of FIGS. 2A to 2C positioned in a user's vagina, according to an aspect of the present disclosure.

FIG. 5A illustrates a perspective view of a third example implementation of the medical device of FIG. 1 that includes an applicator, according to an aspect of the present disclosure.

FIG. 5B illustrates an exploded view of the third example implementation of FIG. 5A, according to an aspect of the present disclosure.

FIG. 5C illustrates a front perspective view of the third example implementation of FIG. 5A with the body of the medical device deployed from the applicator, according to an aspect of the present disclosure.

FIG. 5D illustrates a back perspective view of the third example implementation of FIG. 5A with the body of the medical device deployed from the applicator, according to an aspect of the present disclosure.

Like reference numbers and designations in the various drawings indicate like elements.

### DETAILED DESCRIPTION

Transcutaneous electrical nerve stimulation (TENS) is a non-invasive, inexpensive, self-administered technique to relieve pain that is based on the gate control theory of abolishing the painful stimuli by providing simultaneous inputs in larger myelinated nerve fibers. There are few side effects and no potential for overdose so patients can titrate the treatment as required. The TENS techniques include conventional TENS, acupuncture-like TENS and intense TENS. The purpose of conventional TENS is to selectively activate large diameter non-noxious afferents (A-beta) to reduce nociceptor cell activity and sensitization at a segmental level in the central nervous system. In this way, TENS provides advantages over typical chronic pain treatments.

The present application describes various exemplary embodiments of intravaginal medical devices and methods for treating chronic pelvic pain that utilize TENS. The provided medical device includes at least one electrode such that the medical device may be used as a TENS unit. Stated differently, the provided medical device is capable of supplying electrical pulses to modulate nerves. Chronic pelvic pain can be inhibited by direct inhibition of impulses in the preganglionic afferent neuron by closing the hypothetical gate in the dorsal horn of the spinal cord. The provided medical device may include an arrangement of electrodes (e.g., at least one electrode) such that, while the device is implanted in a user's vagina, the provided medical device may selectively modulate specific nerve endings to selectively modulate different types of pain. In at least some embodiments, the provided medical device may both: (1) target a source of pain and (2) control bleeding during a menstrual cycle. In such embodiments, the body of the provided medical device includes an absorbent material (e.g., cotton) capable of absorbing menses.

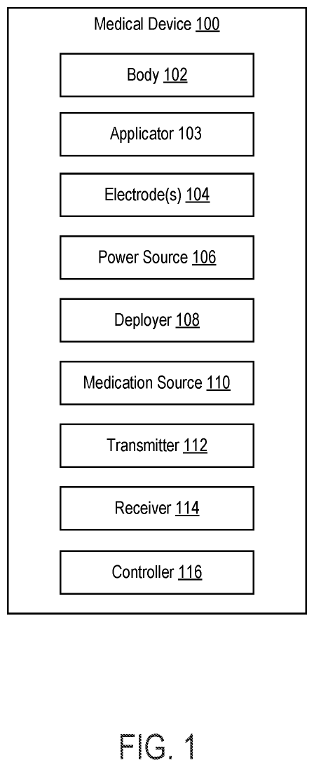

FIG. 1 illustrates a block diagram of an example medical device 100 of the present application. Medical device 100 includes a body 102 that is structured to anatomically fit, and be positioned within, a user's vagina. In at least some aspects, body 102 of medical device 100 may have a shape that is anatomically accurate to the female posterior fornix such that while medical device 100 is positioned within a user's vagina, medical device 100 is snug to the back of the user's cervix. In some aspects, body 102 is a single component. In other aspects, body 102 may be two or more separate components coupled together. In some aspects, medical device 100 may include an applicator 103 that is utilized to deploy medical device 100 into the user's vagina.

Medical device 100 includes one or more electrodes 104. A power source 106 supplies electricity to electrode(s) 104 so that electrode(s) 104 are capable of supplying electrical pulses to objects in contact with electrode(s) 104. Power source 106 may be a battery or another suitable energy source. In at least some aspects, power source 106 is positioned within body 102 of medical device 100 such that body 102 houses power source 106. Electrode(s) 104 may be integrated with body 102 or attached to body 102. In aspects in which body 102 is two or more separate components, a single component may include all of the electrode(s) 104 or two or more components may each include a portion of the electrode(s) 104.

In an example usage scenario, medical device 100 may be used as a TENS unit to supply electrical pulses to one or more nerves to modulate the one or more nerves and inhibit pain felt by a user. For instance, chronic pelvic pain can be inhibited by direct inhibition of impulses in the preganglionic afferent neuron by closing the hypothetical gate in the dorsal horn of the spinal cord. Modulating nerves, as used herein, refers to the alteration of nerve activity through targeted delivery of electrical impulses via electrode(s) 104. Electrode(s) 104 may be arranged such that, while medical device 100 is implanted in a user's vagina, medical device 100 may selectively modulate specific nerve endings via electrode(s) 104 to selectively modulate different types of pain. For example, in at least some embodiments, medical device 100 is shaped so that when medical device 100 is positioned in a user's vagina and activated, medical device 100 applies electrical stimulation to the user's hypogastric nerve plexus, pelvic splenic nerves, or both, around the uterosacral ligaments and behind the cervix. In such embodiments, electrode(s) 104 of medical device 100 are arranged to supply electrical pulses at multiple locations around the cervix. For example, medical device 100 may cradle the user's uteral sacral when so positioned in such embodiments.

Medical device 100 may apply electrical pulses via electrode(s) 104 at various frequencies (i.e. quantity of electrical pulses per second). In some instances, the device may apply electrical pulses at a frequency within a range of 80 to 120 cycles per second. Such instances may help manage acute pain. In other instances, the device may apply electrical pulses at a frequency within a range of 1 to 20 cycles per second. Such other instances may be suitable for the treatment of chronic pain.

In at least some embodiments, medical device 100 includes a deployer 108, such as a string. In such embodiments, activating deployer 108 (e.g., pulling deployer 108) activates power source 106 to supply electricity to electrode(s) 104 so that electrode(s) 104 apply electrical pulses.

As described above, medical device 100 may be used to inhibit pain. In some embodiments, medical device 100 may also be used to control bleeding during a menstrual cycle. In such embodiments, body 102 of medical device 100 includes an absorbent material capable of absorbing menses. For example, body 102 includes a tampon in one implementation of medical device 100. In some aspects, electrode(s) 104 are integrated with, or attached to, the tampon. Stated differently, in such aspects, body 102 may be a single component that is a tampon. In other aspects, electrode(s) 104 are integrated with or attached to a component that is coupled to the tampon. For example, the tampon may be a first component of body 102 and a second component of body 102 includes electrode(s) 104.

In other embodiments, medical device 100 may be structured so that medical device 100 can be used with a separate apparatus that is used to control bleeding during a menstrual cycle. For example, medical device 100 may be designed so that body 102 can be coupled to a tampon as part of a system that includes medical device 100 and the tampon.

In some embodiments, medical device 100 may include a medication source 110 such that medical device 100 can secrete medication (e.g., anti-anxiety medications, NSAIDs, antidepressant medication, cannabis). Medication source 110 can be housed within body 102 or otherwise coupled to body 102. In such embodiments, body 102 may include a porous surface so that medication from medication source 110 can secrete out of body 102 and into the user. In various aspects, the porous surface dissolves throughout use of medical device 100 within a user's vagina to deliver a calculated, time-released amount of the medication from medication source 110. The secreted medication could, for example, be used for pain relief, premenstrual syndrome (PMS), or post-partum depression.

In some embodiments, medical device 100 may include a transmitter 112 and a receiver 114 that enables medical device 100 to communicate with a separate device, such as an application (“app”) on a smartphone. In such embodiments, a controller 116 of medical device 100 may transmit and receive information to and from the separate device via transmitter 112 and receiver 114. For example, a user may select a button on the user's smartphone to initiate (or adjust the frequency of) electrical pulses supplied by electrode(s) 104. The smartphone transmits a signal to receiver 114. Controller 116 receives the signal via receiver 114 and controls power source 106 to supply electricity to electrode(s) 104 according to the signal. In some aspects, controller 116 may transmit to the smartphone via transmitter 112 an acknowledgement that the electrical pulses have been initiated or adjusted. Medical device 100 and the separate device may communicate via Bluetooth® or another suitable communication protocol. In another example, medical device 100 may include a sensor that detects how saturated an absorbent material (e.g., tampon) of medical device 100 is when positioned within a user's vagina, and a signal is sent via transmitter 112 to the user's smartphone such that the user is notified in the app of the saturation level. In some aspects, medical device 100 may include a transceiver instead of a separate transmitter 112 and receiver 114. Controller 116 may include a processing system that includes one or more processors coupled to one or more memories storing processor-executable code. The processing system enables controller 116 to perform the operations described above as well as other similar operations.

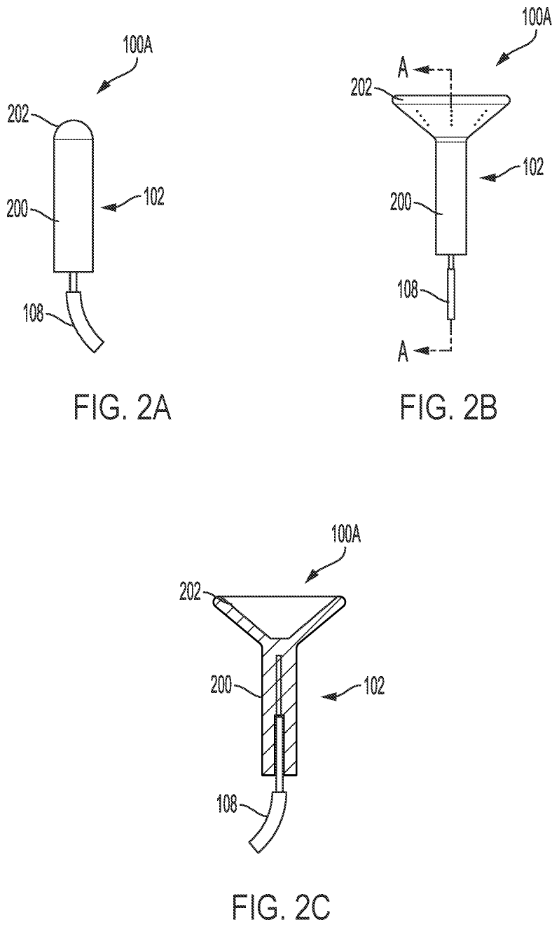

Body 102 of medical device 100 may have one of a variety of suitable shapes that are anatomically accurate to the female posterior fornix such that, while medical device 100 is positioned within a user's vagina, medical device 100 is snug to the back of the user's cervix and electrode(s) 104 contact at least a portion of (e.g., all of) the “u-shape” of the uterosacral nerve. Stated another way, electrode(s) 104 contact the user alongside the outside of the user's cervix as well as the user's distal vagina. In this way, electrode(s) 104 are positioned to apply electrical stimulation to the user's hypogastric nerve plexus or pelvic splenic nerves, or both, around the uterosacral ligaments and behind the cervix. Spanning across the uterosacral ligaments with electrode(s) 104 can help numb the back of the uterus when electrical stimulation is applied to aid in easing menstrual cramps. FIGS. 2A to 2C, 3A and 3B, and 5A to 5C described below each illustrate an alternative, non-limiting example implementation of body 102, as indicated by the same reference numeral being used throughout these figures.

For example, FIGS. 2A to 2C illustrate a medical device 100A that is a first example implementation of medical device 100 with a cone-shaped design. FIG. 2A shows medical device 100A having a first portion 200 of body 102 and a second portion 202 of body 102. In some aspects, first portion 200 may be integral with second portion 202. In other aspects, first portion 200 may be coupled with second portion 202. In some aspects, one or both of first portion 200 and second portion 202 may include an absorbent material (e.g., cotton). For example, first portion 200 may be or include a tampon. Each of first portion 200 and second portion 202 may be reusable or disposable in various aspects. In FIG. 2A, second portion 202 is a cone-shaped portion shown in a closed position. Medical device 100A may be inserted into a user's vagina when cone-shaped second portion 202 is in the closed position. FIG. 2B shows medical device 100A with cone-shaped second portion 202 expanded in a deployed position. Cone-shaped second portion 202 can be deployed from the closed position after insertion of medical device 100A. For instance, cone-shaped second portion 202, in this example, is deployed by pulling deployer 108 away from body 102. For example, medical device 100A may include multiple prongs that provide support to the cone-shaped second portion 202, and may include a deployment mechanism having strings coupled to both the deployer 108 and the multiple prongs at the distal end of cone-shaped second portion 202. In a closed position, the prongs of the cone-shaped second portion 202 are straight and uniform. When the deployer 108 is pulled away from body 102, the strings coupled to the multiple prongs of the distal end are pulled to expand the distal ends of the prongs away from one another.

In some aspects, pulling deployer 108 activates power source 106 to supply electricity to electrode(s) 104. Electrode(s) 104 are integrated with or attached to second portion 202 in this example implementation. For example, electrode(s) 104 are disposed on the inside and outside of deployed cone-shaped second portion 202. For example, electrode(s) 104 may be spaced apart around the circumference of second portion 202, such as at positions of 2, 4, 8, and 10 with reference to a clock with 12 being the front of the user's body and 6 being the back of the user's body. With medical device 100A properly inserted into a user's vagina and second portion 202 deployed, second portion 202 (and therefore electrode(s) 104 of second portion 202) surrounds the user's cervix. In this way, electrode(s) 104 are positioned to apply electrical stimulation to the user's hypogastric nerve plexus, pelvic splenic nerves, or both, around the uterosacral ligaments and behind the cervix. Spanning across the uterosacral ligaments with electrode(s) 104 can help numb the back of the uterus when electrical stimulation is applied to aid in easing menstrual cramps.

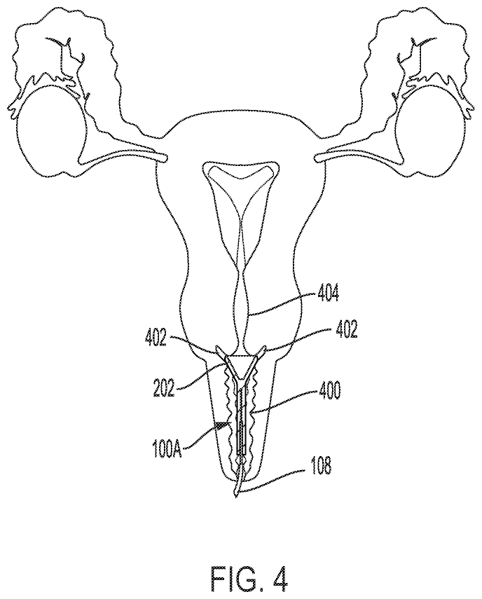

For example, FIG. 4 illustrates a schematic of medical device 100A being positioned within a vagina 400 for use. As shown, the cone-shaped second portion 202 surrounds the cervix 404. The shape of second portion 202 additionally matches (e.g., is anatomically accurate to) the posterior fornix 402 such that second portion 202 is snug to the back of the cervix 404.

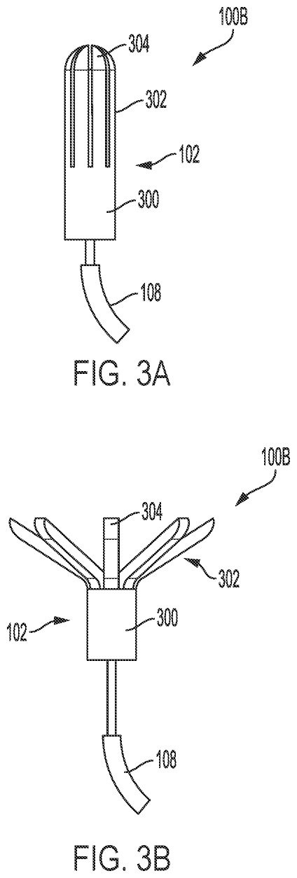

FIGS. 3A and 3B illustrate a medical device 100B, which is a second example implementation of medical device 100. Medical device 100B includes a body 102 with a peacock fan design. FIG. 3A shows medical device 100B having a first portion 300 of body 102 and a second portion 302 of body 102. In some aspects, first portion 300 may be integral with second portion 302. In other aspects, first portion 300 may be coupled with second portion 302. In some aspects, one or both of first portion 300 and second portion 302 may include an absorbent material (e.g., cotton). For example, first portion 300 may be or include a tampon. Each of first portion 300 and second portion 302 may be reusable or disposable in various aspects.

In FIG. 3A, second portion 302 is a peacock fan portion including multiple flexible arms 304 and is shown in a closed position. Medical device 100B may be inserted into a user's vagina when second portion 302 is in the closed position. FIG. 3B shows medical device 100B with flexible arms 304 of second portion 302 expanded in a deployed position. In this example, second portion 302 includes six flexible arms 304, though other suitable quantities of flexible arms 304 may be implemented. Second portion 302 can be deployed from the closed position after insertion of medical device 100B. Second portion 302, in this example, is deployed by pulling deployer 108 away from body 102. For example, medical device 100B may include a deployment mechanism having strings coupled to both the deployer 108 and the distal tips of each of the flexible arms 304. In a closed position, the flexible arms 304 are straight and uniform. When the deployer 108 is pulled away from body 102, the strings coupled to the distal tips of each of the flexible arms 304 in this example deployment mechanism are pulled to expand the distal tips of each of the flexible arms 304 away from one another.

In some aspects, pulling deployer 108 activates power source 106 to supply electricity to electrode(s) 104. Electrode(s) 104 are integrated with or attached to second portion 302 in this example implementation. For example, in some aspects, each arm 304 may include one or more of electrode(s) 104. In other aspects, only a portion of flexible arms 304 include one or more of electrode(s) 104, such as only the flexible arms 304 at positions of 2, 4, 8, and 10 with reference to a clock with 12 being the front of user's body and 6 being the back of user's body. In some aspects, one or more of the electrode(s) 104 may span a portion of the inner surface of the flexible arms 304, such as the entire inner surface. In some aspects, one or more of the electrode(s) 104 may, additionally or alternatively, span a portion of the outer surface of the flexible arms 304, such as the entire outer surface.

With medical device 100B properly inserted into a user's vagina and second portion 302 deployed, second portion 302 (and therefore electrode(s) 104 of second portion 302) surrounds the user's cervix. In this way, electrode(s) 104 are positioned to apply electrical stimulation to the user's hypogastric nerve plexus, pelvic splenic nerves, or both, around the uterosacral ligaments and behind the cervix. Spanning across the uterosacral ligaments with electrode(s) 104 can help numb the back of the uterus when electrical stimulation is applied to aid in easing menstrual cramps.

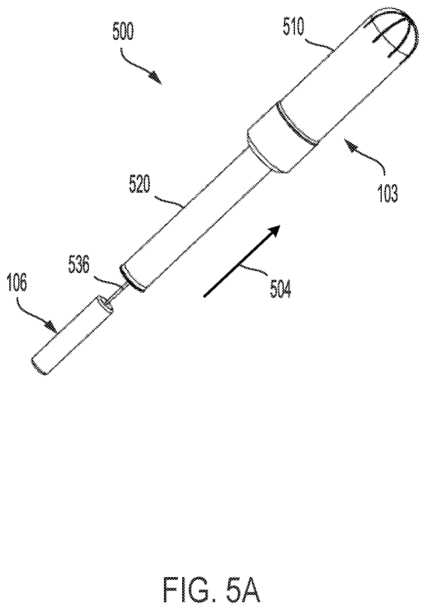

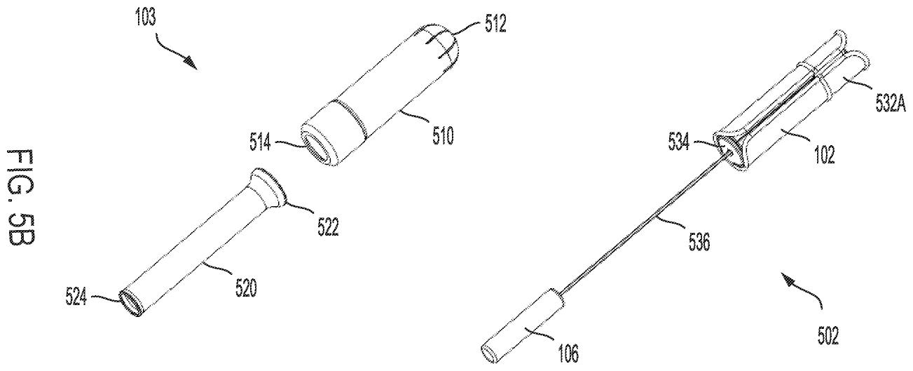

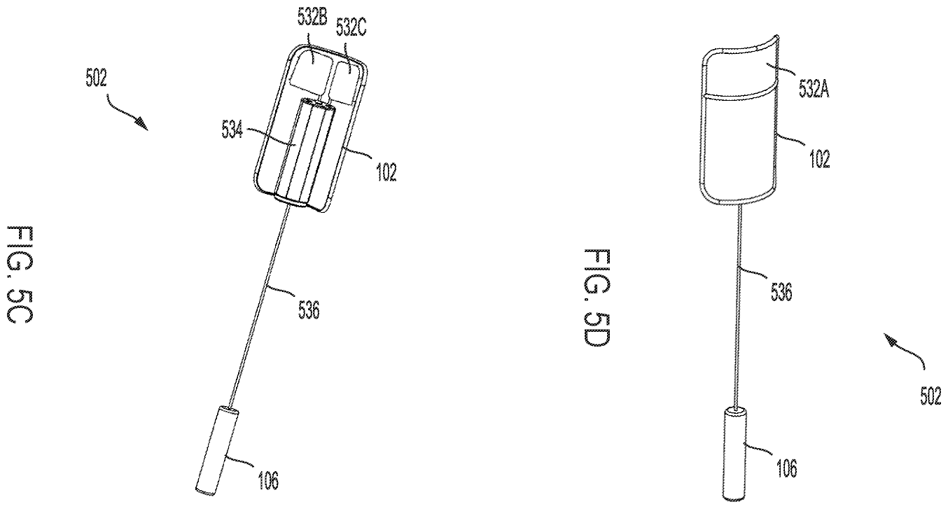

FIGS. 5A to 5D illustrate a medical device 500. Medical device 500 is a third example implementation of medical device 100 that includes a body 102 with a curved design and includes an example implementation of an applicator 103. Medical device 500 is designed to be disposable. The perspective view of FIG. 5A shows medical device 500 in a pre-deployment configuration. The example applicator 103 includes a first portion 510 and a second portion 520. Body 102 of medical device 500 is positioned within the first portion 510 of applicator 103 in the pre-deployment configuration. Sliding the second portion 520 into the first portion 510 along the arrow 504 by applying force to the second portion 520 in the direction of the arrow 504 deploys body 102 out of a first end 512 of the first portion 510. Body 102 is coupled to a power source 106 by a string 536. String 536 may include one or more wires that electrically couple the power source 106 to the electrode(s) 104 of medical device 500. FIGS. 5A to 5D illustrate power source 106 (e.g., a battery) contained within a housing. The housing may include a button, or other activation means, for initiating or terminating the supply of electricity from power source 106. For example, the button may be a mechanical press button or an electrical touch button.

FIG. 5B is an exploded view of medical device 500 showing applicator 103 and a medical device 502. Applicator 103 is shown with the first portion 510 separated from the second portion 520. The first portion 510 includes the first end 512, a second end 514, and a cavity therebetween. The first end 512 may initially be at least partly closed prior to deployment. The second end 514 includes an opening. The second portion 520 includes a first end 522, a second end 524, and a cavity therebetween. As shown, the first end 522 may include a lip so that, when applicator 103 is fully assembled, the first end 522 is positioned within the cavity of the first portion 510 and the lip prevents the second portion 520 from being pulled out of the opening of the second end 514 separated from the first portion 510. The second end 524 includes an opening.

Medical device 502 is deployed by applicator 103 into a user's vagina for use. Body 102 is shown in FIG. 5B as if body 102 is positioned within the cavity of the first portion 510 of applicator 103. Specifically, body 102 is shown in a compressed state. As shown by FIG. 5A, when body 102 is positioned within the cavity of the first portion 510, the string 536 is positioned through, and extends out of, the cavity of the second portion 520 such that power source 106 is external to applicator 103. To deploy medical device 502 from applicator 103, the second portion 520 is pushed into the first portion 510 in the direction of arrow 504 such that the second portion 520 forces body 102 out of the first portion 510 through the opening 512. Once body 102 is clear of the first portion 510 of applicator 103, body 102 expands into an expanded state due to the elastic properties of body 102, which is shown in FIGS. 5C and 5D. Applicator 103 can then be slid back over string 536 and power source 106 and disposed of.

Body 102 is constructed of a medical-grade material (e.g., suitable to be placed within a user's vagina) with suitable elasticity to be compressed within applicator 103 for ease of insertion and deployment in the user's vagina and suitable rigidity to hold a shape when deployed within the user's vagina. For example, body 102 may be constructed of a medical-grade silicone. Body 102 may have a length (e.g., measured along an axis extending along the string 536) that is equal to or less than a length of an average user's vagina.

Electrode(s) 104 of medical device 502 are shown implemented as electrodes 532A, 532B, and 532C that are integrated with, or coupled to, body 102. In some aspects, electrode 532B, electrode 532C, or both, wrap around the edges of body 102 to contact electrode 532A. In some aspects, electrode 532B and electrode 532C contact one another or are combined into a single, integral electrode. In some aspects, electrodes 532A, 532B, and 532C are integral with one another as a single electrode that wraps around the entire end portion of body 102 including the edges.

In at least some aspects, applicator 103 includes an indication of which portion of applicator 103 should face the user's front side and which portion should face the user's back side so that electrodes 532A, 532B, and 532C are positioned properly within the user's vagina when medical device 502 is deployed from applicator 103. The curved shape of body 102 enables body 102, when deployed in the vagina, to be snug to the back of the user's cervix and enables electrodes 532A, 532B, and 532C to contact at least a portion of (e.g., all of) the “u-shape” of the uterosacral nerve. Stated another way, electrodes 532A, 532B, and 532C contact the user alongside the outside of the user's cervix as well as the user's distal vagina. In this way, electrodes 532A, 532B, and 532C are positioned to apply electrical stimulation to the user's hypogastric nerve plexus or pelvic splenic nerves, or both, around the uterosacral ligaments and behind the cervix. When medical device 500 is properly positioned within the user's vagina, power source 106 and a portion of string 536 extend outside of the vagina.

The curved design of body 102 in the implementation of medical device 502 is just one of a variety of shapes possible for body 102, as mentioned above with respect to body 102. For example, body 102 in this implementation may have a shape similar to the cone-shaped design of FIGS. 2A to 2C. In another example, body 102 in this implementation may have a shape similar to the peacock fan design of FIGS. 3A and 3B.

In some aspects, body 102 may include, or be coupled to, an absorbent material 534 (e.g., cotton) for absorbing menses. For example, the absorbent material 534 of body 102 may include, or be, a tampon. In some aspects, as shown in FIG. 5C, body 102 may be coupled to an exterior of absorbent material 534. In such aspects, absorbent material 534 provides structural support to body 102 and vice versa. In other aspects, body 102 may include a portion that is positioned within, and coupled to, an interior of absorbent material 534. In such aspects, the remaining portion of body 102 extends away from absorbent material 534. For example, body 102 may have a trunk portion positioned within, and coupled to, the interior of absorbent material 534 and flexible arms of a peacock fan design extending from the trunk portion. In another example, body 102 may have a portion of cone-shaped design extending from the trunk.

In at least some aspects, after medical device 502 is deployed within the user's vagina, body 102 (and therefore electrodes 532A, 532B, and 532C) are maintained in position by suction between the material (e.g., medical-grade silicone) of body 102 and the user. To remove medical device 502, the user pulls on string 536 until medical device 502 is removed from the vagina. During removal, body 102 collapses inward for easy removal.

An example method for treating chronic pelvic pain includes positioning a medical device (e.g., medical device 100) within a vagina of a user. Electrical pulses (e.g., transcutaneous electrical nerve stimulation) may then be applied to the hypogastric nerve plexus of the user via one or more electrodes (e.g., electrode(s) 104) of the positioned medical device 100. In some aspects, the electrical pulses are applied in response to activating a deployer (e.g., deployer 108) of medical device 100. In other aspects, the electrical pulses are applied in response to a signal received from a device (e.g., a smartphone, smartwatch, laptop, etc.) in communication with medical device 100. In at least some aspects, the method may further include absorbing menses of the user via medical device 100.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any of the various system embodiments may have been presented as a group of particular components. However, these systems should not be limited to the particular set of components, nor their specific configuration, communication and physical orientation with respect to each other. One skilled in the art should readily appreciate that these components can have various configurations and physical orientations (e.g., wholly separate components, units and subunits of groups of components, different communication regimes between components). In general, any suitable combination of features of the various examples described is contemplated.

The invention is claimed as follows:

1. A medical device comprising:
a body configured such that the body may be positioned within a vagina of a user;
at least one electrode integral with, or coupled to, the body; and
a power source configured to supply the at least one electrode with electricity.

2. The medical device of claim 1, wherein the body includes an absorbent material.

3. The medical device of claim 1 or 2, wherein the body includes a tampon.

4. The medical device of any of the preceding claims, further comprising a deployer, wherein the body includes a plurality of arms configured to interact with the deployer such that, prior to activation of the deployer, the plurality of arms are in a closed position, and subsequent to activation of the deployer, the plurality of arms are in an open position, and wherein the plurality of arms include the at least one electrode.

5. The medical device of claim 4, wherein the plurality of arms are arranged such that, in the open position, the plurality of arms span across the uterosacral ligaments of the user while the medical device is positioned within the vagina of the user.

6. The medical device of any of the preceding claims, further comprising a deployer, wherein the body includes a cone-shaped portion configured to interact with the deployer such that, prior to activation of the deployer, the cone-shaped portion is in a closed position, and subsequent to activation of the deployer, the cone-shaped portion is in an open position, and wherein the cone-shaped portion includes the at least one electrode.

7. The medical device of any of the preceding claims, further comprising a deployer configured such that activating the deployer activates the power source to begin supplying electricity to the at least one electrode.

8. The medical device of any of the preceding claims, wherein the body is shaped such that the at least one electrode contacts the hypogastric nerve plexus of the user while the medical device is positioned within the vagina of the user.

9. The medical device of any of the preceding claims, further comprising (1) a transceiver or (2) a transmitter and a receiver.

10. The medical device of any of claims 1 to 9, further comprising an applicator, wherein the body is positioned within the applicator.

11. The medical device of claim 10, wherein the applicator includes a first portion and a second portion configured to slide within the first portion, and wherein the body is positioned within the first portion.

12. The medical device of claim 11, wherein the body of the medical device includes a first end and a second end, and wherein the body is configured such that, upon deploying the medical device from the first portion, the first and second ends translate away from one another in a plane perpendicular to an axis along which the second portion is configured to slide.

13. A method for treating pelvic pain comprising:
positioning a medical device within a vagina of a user, the medical device including:
a body configured such that the body may be positioned within a vagina of a user,
at least one electrode integral with, or coupled to, the body, and
a power source configured to supply the at least one electrode with electricity; and
applying transcutaneous electrical stimulation to the hypogastric nerve plexus of the user via the medical device positioned within the vagina of the user.

14. The method of claim 13, further comprising absorbing menses of the user via the medical device positioned within the vagina of the user.

15. The method of claim 13 or 14, wherein applying transcutaneous electrical stimulation includes applying electrical pulses at a frequency within a range of 1 to 20 cycles per second.

16. The method of claim 13 or 14, wherein applying transcutaneous electrical stimulation includes applying electrical pulses at a frequency within a range of 80 to 120 cycles per second.

17. The method of any of claims 13 to 16, wherein the body includes a plurality of arms movable between a closed position and an open position, wherein the plurality of arms include the at least one electrode, and wherein positioning the medical device includes:
inserting the medical device into the vagina while the plurality of arms are in the closed position, and
deploying the plurality of arms into the open position subsequent to the medical device being inserted into the vagina.

18. The method of claim 17, wherein positioning the medical device further includes positioning the plurality of arms in the open position such that the plurality of arms span across uterosacral ligaments of the user.

19. The method of any of claims 13 to 16, wherein the body includes a cone-shaped portion movable between a closed position and an open position, wherein the cone-shaped portion includes the at least one electrode, and wherein positioning the medical device within the vagina of the user includes:
inserting the medical device into the vagina while the cone-shaped portion is in the closed position, and
deploying the cone-shaped portion into the open position subsequent to the medical device being inserted into the vagina.

20. The method of claim 19, wherein positioning the medical device further includes positioning the cone-shaped portion in the open position such that the cone-shaped portion surrounds a cervix of the user.

21. The method of any of claims 13 to 20, wherein the medical device further comprises a deployer, and the transcutaneous electrical stimulation is applied in response to activating the deployer.

22. The method of any of claims 13 to 21, wherein positioning the medical device includes positioning the medical device within the vagina such that the at least one electrode contacts the hypogastric nerve plexus of the user.

23. The method of any of claims 13 to 22, wherein the transcutaneous electrical stimulation is applied in response to receiving a signal transmitted wirelessly from a device separate from the medical device.

24. The method of any of claims 13 to 23, wherein positioning the medical device within the vagina of the user includes deploying the medical device from an applicator.

25. The method of claim 24, wherein the body of the medical device includes a first end and a second end, and wherein the body is configured such that, upon deploying the medical device, the first and second ends translate away from one another in a plane perpendicular to a central axis of the applicator.
